(19) United States
(12) Patent Application Publication
(10) Pub. No.: US 2026/0083332 A1
(43) Pub. Date: Mar. 26, 2026

(54) INTERFEROMETRIC NEAR INFRARED SPECTROSCOPY SYSTEM AND METHOD FOR NEUROIMAGING AND ANALYSIS

(71) Applicant: COMIND TECHNOLOGIES LIMITED, London (GB)

(72) Inventor: William PARKER, London (GB)

(21) Appl. No.: 19/109,481

(22) PCT Filed: Sep. 8, 2023
(86) PCT No.: PCT/GB2023/052323

§ 371 Date: Mar. 6, 2025

(30) Foreign Application Priority Data
Sep. 8, 2022 (GB) 2213140.3

(51) Int. Cl. A61B 5/00

(52) U.S. Cl. CPC A61B 5/0082; A61B 5/0075; A61B 5/4064; A61B 5/7225

(57) ABSTRACT

An imaging system comprising: a light source configured to provide wavelength-swept emission of light; a sample delivery channel coupled to the light source and arranged to be coupled to an object to be imaged to direct light from the light source towards said object; a reference channel coupled to the light source for receiving light therefrom; a sample receiving channel arranged to be coupled to the object to be imaged for receiving sample light from the object; a detector coupled to the sample receiving channel for receiving sample light, and coupled to the reference channel for receiving reference light, wherein the detector is arranged to combine the received sample light with the reference light to provide a combined light signal comprising one or more components at a beat frequency between sample light and reference light; and a lock-in amplifier arranged to: (i) receive a detection signal based on the combined light signal and formed of one or more components at different frequencies, (ii) receive a selection signal at a selection frequency, and (iii) to provide one or more output signals indicative of a component of the detection signal at the selection frequency.

### TECHNICAL FIELD

The present disclosure relates to the field of imaging an object. In particular, the present disclosure relates to interferometric near infrared spectroscopy (‘iNIRS’) systems and methods for neuroimaging and analysis.

### BACKGROUND

Near infrared spectroscopy (‘NIRS’) is a spectroscopic method which uses the near infrared region of the electromagnetic spectrum (e.g. between 700 and 2500 nm). NIRS systems can be used to provide non-invasive monitoring of scattering and absorption properties of a medium. Radiation at NIRS wavelengths is less easily absorbed by human skin (and also bones) than some visible light, and so NIRS radiation may penetrate both skin and skull, and penetrate into brain tissue. NIRS may be used as a technique for non-invasive imaging of human brain tissue by monitoring scattering and absorption properties of the NIRS radiation within the brain tissue. NIRS systems typically convert received optical signals into digital representations of those signals. In particular, a photodetector may convert the received optical signal into an electrical signal (current/voltage) which is then converted into a digital signal using one or more digitisers. This arrangement places significant demands on digitizer capabilities for the NIRS system. In particular, increasing digitizer bandwidth can be very expensive, and it can also place further constraints on the size of the system.

### SUMMARY

Aspects of the disclosure are set out in the independent claims and optional features are set out in the dependent claims. Aspects of the disclosure may be provided in conjunction with each other, and features of one aspect may be applied to other aspects.

In an aspect, there is provided an imaging system comprising: a light source configured to provide wavelength-swept emission of light; a sample delivery channel coupled to the light source and arranged to be coupled to an object to be imaged to direct light from the light source towards said object; a reference channel coupled to the light source for receiving light therefrom; a sample receiving channel arranged to be coupled to the object to be imaged for receiving sample light from the object; a detector coupled to the sample receiving channel for receiving sample light, and coupled to the reference channel for receiving reference light, wherein the detector is arranged to combine the received sample light with the reference light to provide a combined light signal comprising one or more components at a beat frequency between sample light and reference light; and a lock-in amplifier arranged to: (i) receive a detection signal based on the combined light signal and formed of one or more components at different frequencies, (ii) receive a selection signal at a selection frequency, and (iii) to provide one or more output signals indicative of a component of the detection signal at the selection frequency. The imaging system is arranged to control the selection signal to be at each of a plurality of different selection frequencies during a first time period (e.g. so that output signals from the lock-in amplifier for the first time period are indicative of components of the combined light signal at each of said plurality of different selection frequencies); and wherein the imaging system is configured to provide imaging of the object based on the output signals from the lock-in amplifier (e.g. based on an indication of the components of the combined light signal therein).

Embodiments may simplify signal processing and conversion circuitry for the imaging system. Requirements for a digitiser of the imaging system may be reduced. For example, the digitiser may be able to operate with a lower digitisation bandwidth. By sweeping through a plurality of different selection frequencies, the lock-in amplifier may effectively provide corresponding functionality to performing a Fast Fourier Transform for detection signals. For example, this may be performed in an analogue domain (e.g. so that the output signal from the LIA is an analogue signal, such as a voltage pulse). The system may include a digitiser configured to digitise this LIA output signal to obtain a digital representation of an FFT of the selection signal, thereby simplifying requirements for the signal conversion and processing circuitry of the imaging system.

The lock-in amplifier may comprise a dual phase lock-in amplifier. The lock-in amplifier may comprise a first input port arranged to receive the detection signal; a second input port arranged to receive the selection signal; a first output port arranged to provide an output indicative of an in-phase component of the detection signal; and a second output port arranged to provide an output indicative of a quadrature component of the detection signal. The lock-in amplifier may further comprise at least one of: a third output port arranged to provide an output indicative of a magnitude of the detection signal; and a fourth output port arranged to provide an output indicative of a phase associated with the detection signal.

The imaging system may comprise a controller configured to control the selection signal applied to the lock-in amplifier. The controller may be configured to control the selection signal to sweep through a plurality of selection frequencies during the first time period. The controller may be configured to control at least a portion of the frequency sweep of the selection signals to be non-linear. The controller may be configured to control the selection frequency sweep to sweep through a first frequency range at a greater speed than for a second frequency range. For example, at a first sweep speed (for the first range), the sweep may cover a greater range of frequencies per unit time than for a second sweep speed (for the second range). The controller may be configured to control the selection signal applied to the lock-in amplifier so that, during at least a portion of the first time period, the selection signal is a linearly-chirped sinusoidal signal (e.g. in which the frequency changes linearly). The controller may be configured to control operation of the system to switch between a higher resolution (e.g. slower) mode and a lower resolution (e.g. quicker) mode. The controller may be configured to apply a slower selection frequency sweep in the higher resolution mode than when in the lower resolution mode. The controller may be configured to provide a higher sweep rate for sweeping through the selection frequencies when operating in the quicker mode.

The system may comprise two or more lock-in amplifiers. Each of the lock-in amplifiers may be arranged to operate in a different portion of the frequency spectrum. Each of the lock-in amplifiers may be arranged to sweep its selection signals through a different subset of selection frequencies. The system may be configured to image the object based on results from each of the lock-in amplifiers. The detection signal and the selection signal may be analogue signals. The detection signal and the selection signal may be voltages. The system may comprise one or more electrical signal converters (e.g. photo detectors) arranged to provide the detection signal to the lock-in amplifier, wherein said converters (photo detectors) may be arranged to convert the combined light signal into an electrical signal to provide the detection signal. Each said photodetector may comprise a photodiode. The imaging system may comprise a second detector arranged to receive sample light from the object to be imaged and reference light from the light source. The second detector may be coupled to the reference channel to receive reference light therefrom. The system may comprise a second light source. The detector may be arranged to receive: (i) second sample light which was emitted by the second light source and directed towards the object to be imaged, and (ii) second reference light from the second light source via a second reference channel.

The imaging system may be an interferometric near infrared spectroscopy, iNIRS, system. The imaging system may be a neuroimaging and analysis system for imaging a subject's brain tissue. The sample delivery channel may be arranged to be coupled to the subject's scalp for directing light towards their brain tissue. The sample receiving channel may be arranged to be coupled to the subject's scalp for receiving light from their brain tissue.

In an aspect, there is provided an imaging method comprising: providing wavelength-swept emission of light from a light source; directing light from the light source: (i) towards an object to be imaged, and (ii) to a reference channel; receiving, at a detector, reference light from the reference channel and sample light from the object to be imaged, wherein the received sample light comprises light delivered towards the object from the light source; optically combining sample light with reference light to provide a combined light signal comprising one or more components at a beat frequency between sample light and reference light; providing, to a lock-in amplifier: (a) a detection signal based on the combined light signal and formed of one or more components at different frequencies, and (b) a selection signal at a selection frequency, and wherein the lock-in amplifier is configured to provide one or more output signals indicative of the component of the detection signal at the selection frequency; controlling the selection signal to be at each of a plurality of different selection frequencies during a first time period (e.g. so that output signals from the lock-in amplifier for the first time period are indicative of components of the combined light signal at each of said plurality of different selection frequencies); providing imaging of the object based on the output signals from the lock-in amplifier (e.g. based on the components of the combined light signal indicated therein).

Aspects of the present disclosure may provide one or more computer program products comprising computer program instructions configured to program a controller to control operation of an imaging system to perform any of the methods disclosed herein.

Example imaging systems of the present disclosure may comprise (e.g. be formed of) a light emitting arrangement and a light detecting arrangement. The light emitting arrangement may be configured to: deliver the light towards the object to be imaged (e.g. for delivering sample light to the object via a sample delivery channel), and provide reference light to a reference channel (e.g. for ultimately combining with the sample light at a detector). The light emitting arrangement may comprise the light source, the sample delivery channel and (at least a portion of) a reference channel. The light detecting arrangement may be configured to receive both sample light from the object to be imaged (e.g. via a sample receiving channel) and reference light for combining with the sample light (e.g. via a reference channel). The light detecting arrangement is configured to combine the sample and reference light and to detect beat frequencies in the combined signal thereof. The light detecting arrangement may comprise the sample receiving channel (at least a portion of) a reference channel, and a detector (for optically combining sample and reference light and detecting beat frequency components therein). The detector may comprise a photodetector. The photodetector may be configured to generate an electrical signal based on the combined light signal. The imaging system may comprise signal processing and/or conversion circuitry for converting such electrical signals into digital representations thereof.

Embodiments may provide iNIRS systems and methods for neuroimaging and analysis of a subject's brain tissue. The iNIRS systems of the present disclosure may also include signal processing and conversion circuitry arranged to receive output signals from the one or more light detectors and to convert the output signals into digital representations thereof. The iNIRS systems of the present disclosure are configured to provide neuroimaging and analysis of a subject's brain tissue based such digital representations.

Each light source may comprise a light generating element arranged to generate light (e.g. near infrared light). For example, each light generating element may comprise a laser. Each light source may comprise an optical arrangement coupled to the light generating element. The optical arrangement of each light source may be configured to deliver the generated light from the light generating element to each of one or more different locations. The optical arrangement of each light source may comprise a light splitter for splitting light into each of the different delivery light channels. The iNIRS system may be arranged so that, when installed on a subject's head (e.g. for providing neuroimaging and analysis of that subject's brain tissue), the optical arrangement of each light source may be configured to deliver both: (i) light to the sample light delivery channel (‘sample light’), and (ii) light to the reference delivery channel (‘reference light’). Each light source may be arranged to provide (e.g. wavelength-swept) emission of light (e.g. each light source may be arranged to output light at each of a plurality of different wavelengths in a selected time period). Each light source may be arranged to sweep the wavelength of the light it outputs (e.g. increasing or decreasing in wavelength). Each light source may be arranged to provide chirped emission of light in which, each chirp (or ‘pulse’) comprises one wavelength sweep. Each light source may be arranged to output sequential chirps with the same wavelength sweep, e.g. such that the wavelength of the light output from the light source changes according to a repeating pattern.

Each light detector may provide an interferometric optical detector. Each light detector may comprise an optical arrangement. The iNIRS system may be arranged so that, when installed on a subject's head (e.g. for providing neuroimaging and analysis of that subject's brain tissue), the optical arrangement of the light detector is configured to receive sample light from each light source (e.g. light which was emitted from the light source and which has travelled through the subject's brain tissue) and reference light from each light source which has travelled directly from that light source (e.g. along an optical channel). The light detector may be arranged to combine reference light with sample light to provide a combined light signal. For example, the light detector may comprise a light combiner (e.g. for combining light on the reference receiving channel with light on the sample receiving channel). The combined light signal may include a plurality of components at beat frequencies, e.g. at frequencies corresponding to the differences in wavelength between the sample light and the reference light. Each light detector is configured to convert received combined light signals into one or more electrical signals indicative of that combined light signal. For example, the detector may comprise one or more photodiodes. Each photodiode may output an electrical signal (e.g. a current) indicative of the combined light signal. The detector may comprise a balanced photodetector (e.g. which includes two photodiodes, which may be 180° out of phase with each other, and its output may be a combination of the two photodiode current outputs). The detector may optionally include current to voltage conversion circuitry and/or one or more amplifiers for amplifying the electrical signal.

The iNIRS system may include at least one analogue to digital converter arranged to convert electrical signals representing the sample light (e.g. the combined light signals) into one or more digital signals. The controller is arranged to process the digital signals to determine one or more properties of the subject's brain tissue. The controller may be configured to determine optical properties of the subject's brain tissue (e.g. for absorption and/or scattering). The controller may be configured to determine one or more dynamic properties of the subject's brain tissue (e.g. properties of the subject's brain tissue which are varying over time). For example, the controller may be configured to detect the presence of movement within the subject's brain tissue (e.g. due to movement, such as flow, of blood within the brain tissue).

The controller may be configured to process the digital signals to obtain time of flight information for photons of sample light travelling from each light source through the subject's brain tissue to the light detector. The controller may be configured to identify penetration depths (and optionally expected trajectories for photons through the brain tissue) associated with the different times of flight for sample light photons. The controller may be configured to obtain a time-ordered series of time of flight distributions for sample light photons reaching each light detector. The controller may be configured to process the time-ordered series to identify changes in the time of flight distribution over time, such as identifying decay and/or decay rates between success time of flight distributions. The controller may be configured to provide depth-resolved processing, e.g. by filtering the time of flight data to focus on only photons within a selected time of flight range (e.g. to identify changes in optical properties of the brain tissue for penetration depth(s) associated with that time of flight range). The controller may be configured to process data received from the light detector(s) to provide time of flight information with depth-resolved autocorrelations for the subject's brain tissue.

The controller may be configured to process the received data indicative of sample light received at a light detector and to output a control signal based on that received data. The control signal may provide an indication of the time of flight distribution (e.g. the controller may be configured to output the time of flight distribution). The control signal may provide an indication of one or more properties determined based on the time of flight distribution, such as optical properties for the brain tissue (e.g. scattering and/or absorption coefficients, and/or how these have changed/are changing). The control signal may provide an indication of blood flow within the subject's brain tissue. The control signal may provide a depth-resolved indication of one or more properties of the subject's brain tissue (e.g. linked to a specific region within their brain tissue, such as at a selected penetration depth range). The control signal may comprise an indication of one or more properties of the subject's brain tissue, such as intracranial pressure, blood flow index, artery elasticity, cerebral metabolic rate of oxygen consumption. The medical properties may be associated with specific regions/depths within the subject's brain tissue. The control signal may comprise an actuation command for a brain-computer interface, e.g. to control operation of a device based on the actuation command. The control signal may comprise an image for display, where that image represents a portion of the subject's brain tissue (as determined based on the received sample light).

### FIGURES

Some examples of the present disclosure will now be described, by way of example only, with reference to the figures, in which:

FIG. 1 is a schematic diagram of an interferometric near infrared spectroscopy (‘iNIRS’) system.

FIG. 2 is a schematic diagram illustrating operation of a lock-in amplifier (‘LIA’).

FIGS. 3 to 7 are schematic diagrams of LIAs.

In the drawings like reference numerals are used to indicate like elements.

### SPECIFIC DESCRIPTION

Embodiments are directed to an imaging system which uses a lock-in amplifier (‘LIA’) to facilitate signal processing of received optical signals. The LIA receives two signals. One signal is a detection signal based on light which has passed through an object to be imaged. The detection signal contains components at a plurality of different frequencies. Each component (and its associated frequency) may provide time of flight information for sample light which has travelled through the object. The other signal provided to the LIA is a selection signal at a selection frequency. Both of these signals may be analogue signals, such as voltages. The LIA provides an output signal which corresponds to the component of the detection signal at the selection frequency. The system is operated so that a plurality of different selection frequencies are provided to the LIA during a first time period. The LIA will therefore provide a corresponding plurality of output signals during the first time period, where each output signal corresponds to the component of the detection signal at the respective selection frequency that was applied to the LIA. The selection frequencies that are used are chosen to span a frequency range at which the detection signal may contain relevant time of flight information for imaging the object.

Embodiments may find particular utility as part of an interferometric near infrared spectroscopy (‘iNIRS’) system. The iNIRS system may be particularly well suited for providing neuroimaging and analysis of a subject's brain tissue. The detection signal to be provided to the LIA may contain sample light which has travelled through the subject's brain tissue. The output signals from the LIA at different selection frequencies may be used to provide a time of flight distribution for sample light photons (some of which have travelled through the subject's brain tissue). The iNIRS system may be configured to provide neuroimaging and analysis for the subject's brain tissue based on one or more of these time of flight distributions.

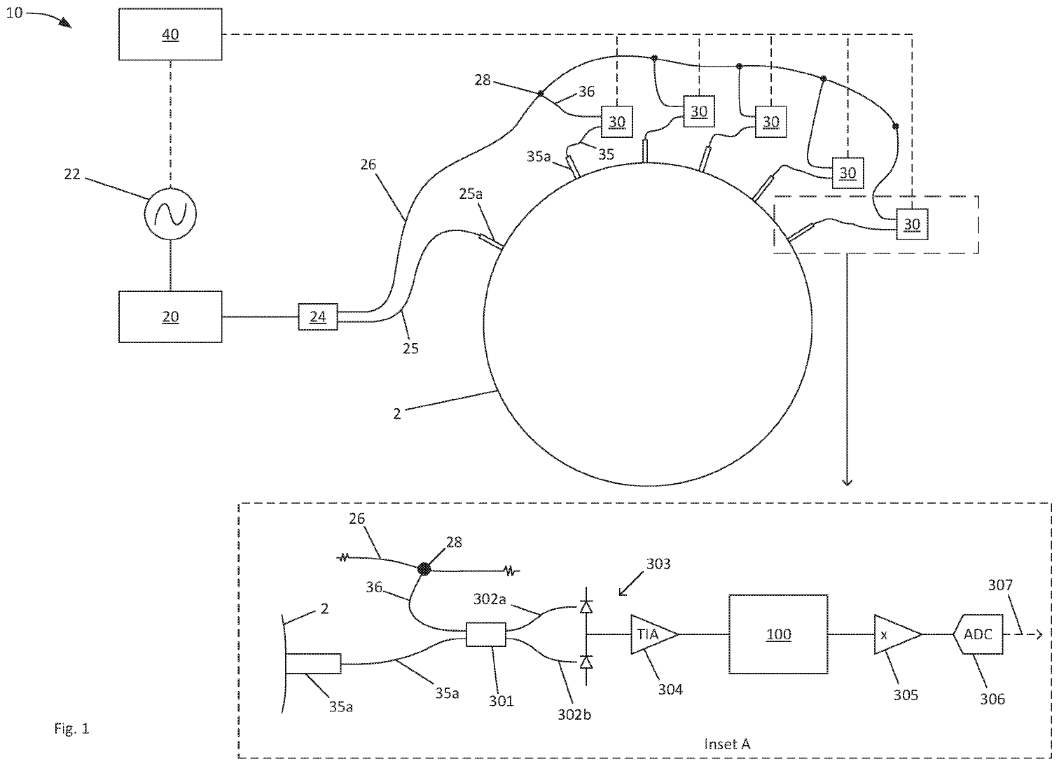

An example of an iNIRS system will now be described with reference to FIG. 1.

Interferometric Near Infrared Spectroscopy (‘iNIRS’)

FIG. 1 shows a schematic diagram of an interferometric Near Infrared Spectroscopy (‘iNIRS’) system 10. The iNIRS system 10 includes a light source 20, a plurality of light detectors 30, and a controller 40. Inset A of FIG. 1 shows a more detailed view of one of the light detectors 30, as well as signal processing circuitry for that light detector.

The iNIRS system 10 includes a light source modifier 22, and a light splitter 24. The iNIRS system 10 includes a sample delivery channel 25 and a reference delivery channel 26. The iNIRS system 10 is shown coupled to a subject's head 2. The iNIRS system 10 includes a sample delivery probe 25a and a plurality of sample receiving probes 35a. For each light detector 30, there is an associated sample receiving probe 35a, a sample receiving channel 35, a reference delivery channel connection 28, and a reference receiving channel 36.

The light source modifier 22 may comprise a source for providing a variable electrical control signal (e.g. a variable current or voltage provider). The light source modifier 22 is coupled to the light source 20. The light source modifier 22 may be electrically connected to the light source 20 to provide a variable current/voltage thereto. The light source 20 may comprise a laser. The light source 20 is coupled to the light splitter 24. The light splitter 24 has an input for receiving light from the light source 20. The light splitter 24 has two outputs for transmitting light from the light source 20 to two separate channels. The sample delivery channel 25 is coupled to the light splitter 24 (to receive light therefrom), as is the reference delivery channel 26. The sample delivery channel 25 couples the light splitter 24 to the sample delivery probe 25a. The sample delivery probe 25a will be placed at a location on the subject's scalp.

The reference delivery channel 26 couples the light splitter 24 to each of the light detectors 30. For each detector, the reference delivery connection 28 couples the reference delivery channel 26 to the reference receiving channel 36 for that detector. Each reference receiving channel 36 is coupled to its light detector 30. Each light detector 30 is connected to the light source 20 to receive reference light therefrom (via one or more reference channels). Each sample receiving probe 35a is placed on the subject's scalp. Each sample receiving probe 35a is coupled to its light detector 30 via its sample receiving channel 35. Each light detector 30 is connected for indirectly receiving sample light from the light source 20 (via the sample delivery and receiving channels, and via the subject's brain tissue therebetween).

There are a plurality of different light detectors 30 shown in FIG. 1. Each detector may provide an interferometer (in combination with the system 10), such as a Mach-Zehnder interferometer. Each of the different light detectors 30 is coupled to the same light source 20 (each via one or more reference channels). The light detectors 30 may be spatially separated from the light source 20. The light detectors 30 may also be spatially separated from one another or they may be co-located on a sufficiently similar region of tissue that the received signals can be averaged together. For reference light to reach the light detector(s) 30 from the light source 20, the reference light will travel directly along one or more reference channels. For sample light to reach the light detector 30 from the light source 20, the sample light will travel via the subject's brain tissue. The sample light is delivered to the subject's scalp via one or more delivery channels. The sample light may then pass through the subject's brain tissue, i.e., the object to be imaged, where it will be received and transmitted to a light detector 30 via one or the sample receiving channels 35.

The controller 40 may comprise any suitable component with data receiving and processing functionality. For example, the controller 40 may include at least one Application Specific Integrated Circuit (‘ASIC’). Other examples for the controller 40 may include a Field Programmable Gate Array (‘FPGA’) and/or a Data Acquisition module (‘DAQ’). The controller 40 is coupled to each of the detectors. The controller 40 may be connected to each detector via a wired connection (for receiving electrical signals indicative of detection therefrom), and/or the connection may be wireless (for receiving transmitted data indicative of detection therefrom). The controller 40 is coupled to the light source modifier 22. This connection may be wired or wireless. The iNIRS system 10 may be at least partially housed within a garment for the subject's head 2. For example, the iNIRS system 10 may be provided in a hat/cap which is to be worn by the subject on their head 2. The head garment may be arranged to hold the light source 20 and detectors in a fixed arrangement relative to the subject's scalp. Some or all of the components may be provided with the head garment. For example, the head garment may include a plurality of receiving portions for receiving light source(s) 20 and light detectors 30. Channels connecting the light sources and light detectors 30 may be provided as part of the head garment (e.g. they may be routed through corresponding channel receiving portions of the head garment). The controller 40 may be separate to the head garment (e.g. and connected wirelessly) or it may also be provided as part of the head garment (e.g. by an ASIC within the head garment which may be wire coupled to the detectors and/or light source modifier 22). For example, the garment may be configured to receive the source and detection channels and the probes, with the other components of the system located elsewhere.

Some or all of the channels of the iNIRS system 10 may be provided by optical fibres. Light splitters of the present disclosure may comprise fibre-optic splitters. The iNIRS system 10 may include lenses, reflection and/or refraction devices for beam steering, as relevant. For example, the sample delivery probe 25a may include one or more lenses for spatially distributing sample light from the sample delivery channel 25 towards the subject's brain tissue. As another example, one or more of the sample receiving probes 35a may include a lens for focussing received light into the sample receiving channel 35 connected to that sample receiving probe 35a. As another example, the probes may be bare fibres which have been cleaved and/or polished.

The iNIRS system 10 is arranged to provide a plurality of source-detector pairs for each light source 20. In other words, the iNIRS system 10 is arranged so that each light detector 30 may receive two forms of light: (i) reference light, and (ii) sample light. Each detector is arranged to receive reference light directly from the light source 20 (the reference light will travel from the light source 20 along one or more channels to the light detector 30, e.g. without passing through the subject's brain tissue). Each detector is also arranged to receive sample light which has travelled through the subject's brain tissue (e.g. which has not travelled exclusively through optical channels between the light source 20 and light detector 30 like the reference light).

The detectors 30 are arranged to be positioned on the subject's scalp to provide imaging of a selected region of their brain. The light source 20 is arranged to generate light and to direct this light towards the subject's scalp and the light detectors 30 (via the reference channel(s)). The light splitter 24 is arranged to receive light generated by the light source 20 and to split this light into two channels: (i) towards the subject's scalp using the sample delivery channel 25 and sample delivery probe 25a, and (ii) to the light detectors 30 using the reference delivery channel 26 and reference receiving channels 36. The splitter is configured so that the majority of the light energy is directed towards the subject's scalp. For example, the splitter may be a 90:10 splitter, or a 99:1 splitter. The sample delivery channel 25 is arranged to receive sample light from the splitter, and to deliver this sample light towards the subject's scalp (via the sample delivery probe 25a). The reference delivery channel 26 is arranged to receive reference light from the splitter, and to deliver this sample light to the detectors (via the reference receiving channels 36).

Each of the reference delivery connections 28 is arranged to deliver some of the reference light travelling along the reference delivery channel 26 to one of the reference receiving channels 36. Each of the reference receiving channels 36 is arranged to deliver the reference light to its light detector 30. The sample receiving probe 35a is arranged to receive sample light from the subject's brain tissue. The sample receiving probe 35a may focus the received sample light onto the sample receiving channel 35. The sample receiving channel 35 is arranged to deliver received sample light to its light detector 30. The sample receiving probes 35a may be arranged in close proximity to each other on the subject's scalp.

Each detector is arranged to receive two inputs: (i) reference light directly (via the reference channel but not via the object) from the light source 20, and (ii) sample light indirectly from the light source 20 (e.g. which has travelled via the subject's brain tissue, as well as through their scalp skin and skull). For example, each detector may comprise two or more input ports. A first input port of the detector may be coupled to the reference delivery channel 26 for that detector. A second input port of the detector may be coupled to the sample delivery channel 25 for that detector. The detector is arranged to combine reference light with sample light (as an interferometer). The detector and controller 40 are arranged to determine one or more properties of the subject's brain tissue based on this combination of reference light and sample light (as will be described in more detail below).

The light source 20 is configured to provide wavelength-swept emission of light. For this, the light source 20 may be configured to produce a series of emissions of pulses of light. During each pulse, the wavelength of light may be “swept” through a range of wavelengths. For example, the sweeping may be in the form of a chirped pulse. The controller 40 may be configured to selectively control the wavelength sweeping of the light source 20. The light source modifier 22 is arranged to control the wavelength emission of light from the light source 20. For instance, the light source modifier 22 may be arranged to apply a selected current (or voltage) to the light source 20 to select a wavelength emission from the light source 20.

Light sources of the present disclosure may be configured to provide emission of high coherence light, e.g. substantially coherent light. The iNIRS system of the present disclosure will receive sample light and reference light, both of which originated from the same light source. The light sources of the present disclosure are configured to provide wavelength-swept emission of sufficiently coherent light, such that the sample light and reference light, as received at the detector, will be in relatively similar phase to each other. In other words, the coherence length of the light source may be such that the multiple scattering in the tissue will not reduce the coherence or fringe contrast below a noise floor for the measurement.

The iNIRS system 10 is arranged so that the source-detector path lengths for reference and sample light are different. In other words, the iNIRS system 10 is arranged so that an average, or expected, optical path length for light travelling from the light source 20 to each detector via the subject's brain tissue will be different to the optical path length for light travelling from the light source 20 to said detector via reference channel(s).

As will be appreciated in the context of the present disclosure, photons of sample light which are directed towards the subject's brain tissue may travel from the light source 20 to a light detector 30 via a practically infinite number of different paths. A photon of sample light may undergo a large number of scattering events, and so follow a very tortuous path, between the sample delivery probe 25a and the sample receiving probe 35a. The iNIRS system 10 is arranged to provide neuroimaging and analysis based at least in part on activity in the subject's brain tissue. The time of flight for a sample light photon from light source 20 to light detector 30 will of course increase as the path length it takes increases. As such, a photon which travels a longer path, and penetrates deeper into the subject's brain tissue, will take longer to arrive at the light detector 30. The longer the time of flight for a sample light photon, the deeper that photon is likely to have penetrated into the subject's brain tissue.

The iNIRS system 10 is arranged so that the shortest time of flight for photons of light to travel from light source 20 to light detector 30 will be for photons of reference light travelling along the reference channel(s). The sample light photons will have longer times of flight than this reference light. The sample light photons which penetrate the deepest into the subject's brain tissue are likely to be those which have the longest time of flight to the light detector 30.

The iNIRS system 10 is arranged so that each of the light detectors 30 receives both sample light and reference light, and combines the two using a light combiner. For example, each of the detectors may provide an interferometer assembly (in combination with the sample and reference light channels of system 10) configured to combine the reference light and the sample light to obtain an interference pattern (an interferogram). The resulting interference pattern for light from the light source 20 (as obtained at each detector) may comprise a combined signal having components at beat (or intermediate/difference) frequencies corresponding to the difference in wavelength between: (i) wavelengths of the photons of sample light received at the light detector 30 at a given instance in time, and (ii) the wavelength of photons of reference light received at the light detector 30 at that instance in time. The sample light will include photons at different wavelengths, where each wavelength of sample light will correspond to the time of flight for that photon and its unique path through the tissue (due to the wavelength sweeping of the light source 20). The resulting interferogram will therefore contain a plurality of different beat frequencies (due to the different differences in wavelength). The higher beat frequencies may correspond to photons with higher times of flight (deeper penetrating photons) in the event that the sample path is longer than the reference path.

In other words, each light detector 30 may comprise a light combiner configured to combine the sample and reference light to provide a combined light signal. The iNIRS system 10 may be arranged to process that combined light signal to obtain an indication of the intensity of light incident on the light detector 30 at a given moment in time (e.g. through use of optical heterodyning and/or balanced detection). The iNIRS system 10 is arranged to obtain a plurality of such indications, e.g. the light detector 30 may be arranged to repeatedly obtain indications of the intensity of light incident on the light detector 30. For example, the iNIRS system 10 may be arranged to measure a phase or frequency shift between photons of light in the two inputs to the detector (reference and sample), and to attribute such differences to properties of the intervening brain tissue for the sample light.

One example of an arrangement for converting received light signals into digital data is shown in Inset A of FIG. 1. Inset A shows an arrangement of components that may be used as a light detector 30 of the present disclosure. As also shown in the iNIRS system 10 of FIG. 1, the detector 30 receives two inputs: (i) reference light which has travelled along reference delivery channel 26 and reference receiving channel 36, and (ii) sample light which has been received through the sample receiving probe 35a and delivered to the detector via the sample receiving channel 35. As shown, the detector may include a light combiner and splitter 301, a first light channel 302a and a second light channel 302b, a balanced photodetector 303, a transimpedance amplifier 304, an amplifier 305, an analogue to digital converter (‘ADC’) 306. The ADC 306 is arranged to provide a digital signal output 307. As shown in Inset A of FIG. 1, the detection circuitry also includes a lock-in amplifier (‘LIA’) 100.

The light combiner and splitter 301 is coupled to both the reference receiving channel 36 and the sample receiving channel 35. The light combiner and splitter 301 is arranged to receive both the sample and reference light, and to combine the two to provide a combined light signal. The light combiner and splitter 301 is arranged to split that combined light signal onto two separate channels: the first light channel 302a and the second light channel 302b. For example, this may be a 50:50 split (or there or thereabouts). The first light channel 302a and second light channel 302b are coupled to a balanced photodetector 303. Each light channel directs light towards an associated photodiode of the photodetector 303. The balanced photodetector is arranged to provide an output based on a difference between outputs from the two photodiodes. The photodetector will typically be provided so that the beat signals on each photodiode are 180° out of phase with each other, and so the coherent AC terms will combine positively with each other. The balanced photodetector 303 is arranged to output a current corresponding to the difference between the two photodiode output currents. The balanced photodetector 303 may remove any unwanted DC terms from this signal, such as slow fluctuations emanating from the light source 20 or other common-mode effects such as noise.

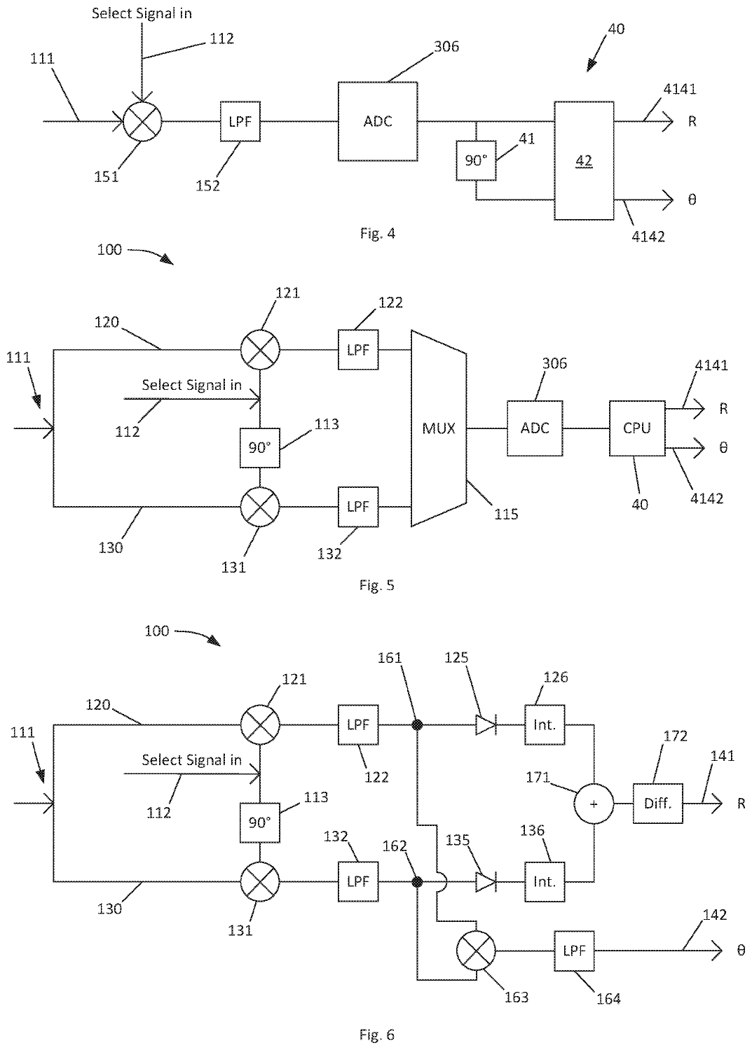

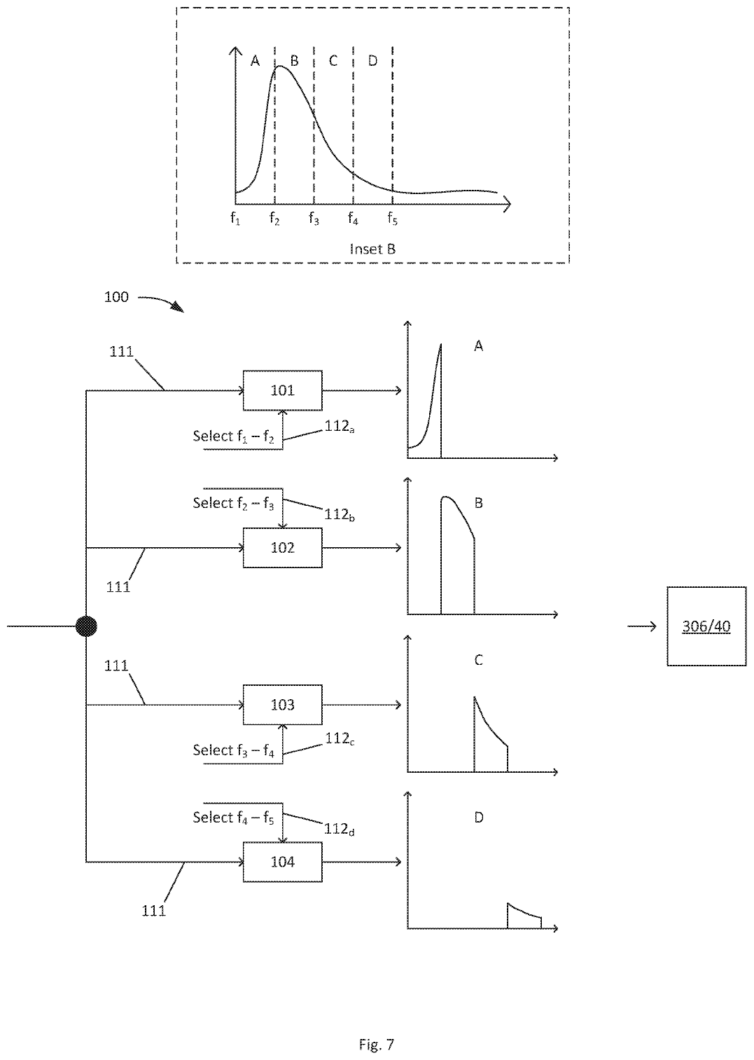

The light detector 30 is configured to use a current to voltage converter to convert the current output from the balanced photodetector 303 into a corresponding voltage. As shown in Inset A of FIG. 1, the converter may comprise a transimpedance amplifier 304. The voltage output from the transimpedance amplifier 304 may then be amplified using the amplifier 305, and/or the amplifier may be provided downstream of the LIA 100 (as shown in FIG. 1), e.g. for amplifying an output signal from the LIA 100. The amplifier may be configured to scale the relevant output signal to the full range of the ADC and limit the electronic frequency of the circuit to further maximise the SNR. This amplified voltage is then provided to the ADC 306 to be digitised. The ADC 306 comprises a digitiser having sufficient bandwidth so that the full signal bandwidth containing relevant time of flight information may be digitised without attenuation. For example, the digitiser bandwidth may be at least as large as the bandwidth for the combined light signal. For example, the digitiser 306 may be selected to have a sampling rate high enough so that the Nyquist criterion is met for the bandwidth of the signal to be processed. Multiple digitisers may be used to improve resolution, as will be discussed with reference to FIG. 6 and FIG. 7. The digitiser may be provided as part of each light detector 30, or the digitiser may be part of the controller 40, and the controller 40 may be coupled to each of the light detectors 30 to receive electrical signals therefrom which are to be digitised. For each combined light signal, a digital signal output 307 will be provided which gives a digital representation based on that combined light signal (and thus of the sample light incident on the light detector 30 associated with that combined light signal).

The iNIRS system 10 is configured to obtain a plurality of digital signal outputs 307 indicative of sample light incident on light detectors 30. In particular, each light detector 30 is configured to repeatedly combine light signals (sample and reference) for providing digital signal outputs 307 representative of each combined light signal. For example, for each light detector 30, a time series of digital signal outputs 307 may be obtained, wherein each subsequent digital signal output 307 is for a subsequent point in time at which a combined light signal was obtained and measured (and the digital signal output 307 represents that combined light signal as obtained and measured). As described in more detail below, each of these signals may be indicative of a sample light time of flight distribution (‘DTOF’) for that point in time at that detector.

In other words, the iNIRS system 10 may be configured to obtain a plurality of time-ordered DTOFs for one or more (e.g. each) of a plurality of light detectors 30. The digitiser may provide a digital output indicative of the different measurements, e.g. in the form of DTOF data containing information about time-ordered DTOF distributions of sample light incident on a detector. The time-ordered series of DTOFs may be considered to correspond to a surface of data in a 3D volume. That surface may represent the DTOF for each subsequent DTOF (ordered in time), and so the surface shows each individual DTOF, as well as the evolution of the DTOFs over time. This surface may provide a wealth of data from which properties of the subject's brain tissue may be determined. An analysis of the temporal fluctuations in (e.g. decay of) DTOF values over time may provide an indication of one or more dynamic properties of the subject's brain tissue. That is, the decay analysis may be used to identify that one or more properties within the subject's brain tissue are changing, as well as optionally identifying the rate at which these properties are changing (e.g. using the decay rate). The order of the decay of DTOF (e.g. decaying with t, t², etc.) may be used to determine properties of the type of motion (e.g. diffusion or flow).

The controller 40 may store data which correlates time of flight for a sample light photon to an indication of average path trajectory for that photon. This may include an indication of the depth of the penetration into the subject's brain tissue for that photon, and/or an indication of the region(s) of the subject's brain tissue through which that photon travelled from light source 20 to light detector 30. The controller 40 may be configured to process the DTOF data by dividing this data up into selected time of flight bins. Within each TOF bin, the data may provide depth-resolved evolution data for the subject's brain tissue. That is, as the TOF may be associated with certain penetration depths or regions, each TOF bin may contain data showing properties associated with a certain penetration depth or region. The evolution of data within each TOF bin may therefore provide an indication of how one or more properties of the subject's brain tissue are evolving. For example, where the evolution suggests a change in movement (e.g. a flow of blood), that movement may be identified, as may the region in which that movement is occurring. For this, a TOF-resolved decay slope may be used to identify how the curve is decaying over time for specific TOFs (e.g. for specific penetration depths/regions).

In other words, the iNIRS system 10 is configured to perform an autocorrelation in which DTOFs for successive wavelength sweeps are combined to assess fluctuations in the light field at the light detector 30 over time. The fluctuations may be quantified due to relevant fluctuations in DTOFs over time. The fluctuations may also be depth-resolved, by identifying the relevant TOFs at which those fluctuations are occurring (and thus the relevant penetration depths/regions).

### Lock-in Amplifier (‘LIA’)

As described above, the present disclosure relates to an imaging system, such as an iNIRS system, which includes a lock-in amplifier as part of a light detecting arrangement for that system. The LIA 100 is arranged to receive two input signals. The LIA 100 may have a first input port and a second input port for receiving the two input signals. A first input port may be arranged to receive a detection signal. A second input port may be arranged to receive a selection signal.

The imaging system includes a detector arranged to generate a combined light signal by combining reference and sample light. The sample light will contain components at a plurality of different wavelengths. The reference light (at any one point in time) will contain light at one wavelength. The combined light signal will include a plurality of components at beat frequencies between the reference wavelength and the plurality of different sample wavelengths. The detection signal provided to the LIA 100 will be indicative of such a combined light signal.

For example, and as described above with reference to FIG. 1, the detector may be coupled to conversion circuitry configured to convert the obtained combined light signal into an electrical signal (e.g. a voltage) representative of that combined light signal. The system is arranged to provide such an electrical signal (e.g. a voltage) to the first input port of the LIA 100. The LIA 100 will receive that electrical signal as the detection signal.

The system may also be arranged to provide a second signal to the LIA 100. The second signal is a selection signal. The selection signal may be an analogue signal, such as a voltage. The selection signal (at any one point in time) may be at a single frequency. For example, the selection signal may only have one component, and that component may oscillate at a selection frequency. The LIA 100 is arranged to receive both the detection signal and the selection signal, and to provide one or more output signals based on the two received input signals (i.e. based on the detection signal and the selection signal). In particular, the LIA 100 is configured to provide, as an output signal, one or more signals indicative of a component within the detection signal at the selection frequency of the selection signal. As mentioned above, the detection signal may be representative of a combination of a plurality of different components, wherein each component oscillates at a respective frequency. The detection signal and its components are based on the combined light signal, comprising one or more components at a beat frequency between sample light and reference light. Each respective frequency may correspond to a beat frequency (where that beat frequency may be indicative of a difference in time of flight between sample and reference light).

The LIA 100 is configured so that, for each selection signal provided to the LIA 100, the LIA 100 will provide one or more output signals indicative of the component of the detection signal which is at the same frequency as the selection frequency of the selection signal. For example, where the selection signal is at a first frequency, the output(s) from the LIA 100 may be indicative of a component within the detection signal which is oscillating at the first frequency.

The system is configured to vary the selection frequency of the selection signal over time. In particular, during a first time period, the system is configured to vary the selection frequency so that the selection signals received at the LIA 100 during the first time period will be at a plurality of different frequencies. For example, during the first time period, the selection signal provided to the LIA 100 may start at a first selection frequency, before that first selection frequency is changed to a second selection frequency. This may occur for a plurality of different selection frequencies. In other words, the selection signal may be at a different selection frequency for different portions of that first time period. For example, the LIA 100 may receive a selection signal throughout the first time period. During the first time period, the selection signal received at the LIA 100 may change in selection frequency.

The system is configured to control the selection signal to be at each of a plurality of different selection frequencies during the first time period. As such, output signals from the LIA 100 may also vary during the first time period, as the component of the detection signal being selected by the selection signal will change (due to the selection frequency of that selection signal changing). The system is configured to select values for the selection frequencies based on expected frequencies for the different components within the detection signal. For example, the system may be configured to sweep the selection signal through a plurality of different selection frequencies in a selection frequency range. That selection frequency range may be selected based on an expected range of beat frequencies for the detection signal. For example, the selection frequency range may be chosen to encompass a portion, or all, of the expected beat frequency spectrum for the detection signal.

As will be appreciated in the context of the present disclosure, for any given object to be imaged, the maximum and minimum expected observable times of flight for sample light photons may be approximately known. For example, there may be a known statistical distribution for modelling photon time of flight. Above an upper time of flight threshold, any sample photons with this time of flight may be statistically unlikely to occur, and/or they may be indistinguishable from sensor noise (if they do occur). Similarly, below a lower time of flight threshold, any sample light photons may be statistically unlikely to occur, and/or if they do occur, they may carry null information, as they represent penetration depths of minimal to no use. The system may be configured to vary the selection frequencies which are provided to the LIA 100 so that the different selection frequencies provided to the LIA 100 will span the majority (or all) of the expected range for beat frequencies.

In other words, the system may be configured to vary the selection frequencies provided to the LIA 100 so that the output signals from the LIA 100 will be indicative of the plurality of different components within the detection signal provided to the LIA 100. For example, the output signals from the LIA 100 will be indicative of each of a plurality of different beat frequency components within the detection signal.

The system may be configured to sequentially provide different selection signals to the LIA 100. Each sequential selection signal may be at a different frequency to the previous selection signal. The LIA 100 may sequentially output signals indicative of the component of the detection signal at the selection frequency provided to the LIA 100. In other words, the LIA 100 may receive a time-ordered series of selection signals, where each selection signal is at a different selection frequency. The LIA 100 may provide a corresponding time-ordered series as the LIA 100 output, where each individual output in the series corresponds to the component of the detection signal at the selection frequency of the selection signal that was applied to the LIA 100 to provide that output.

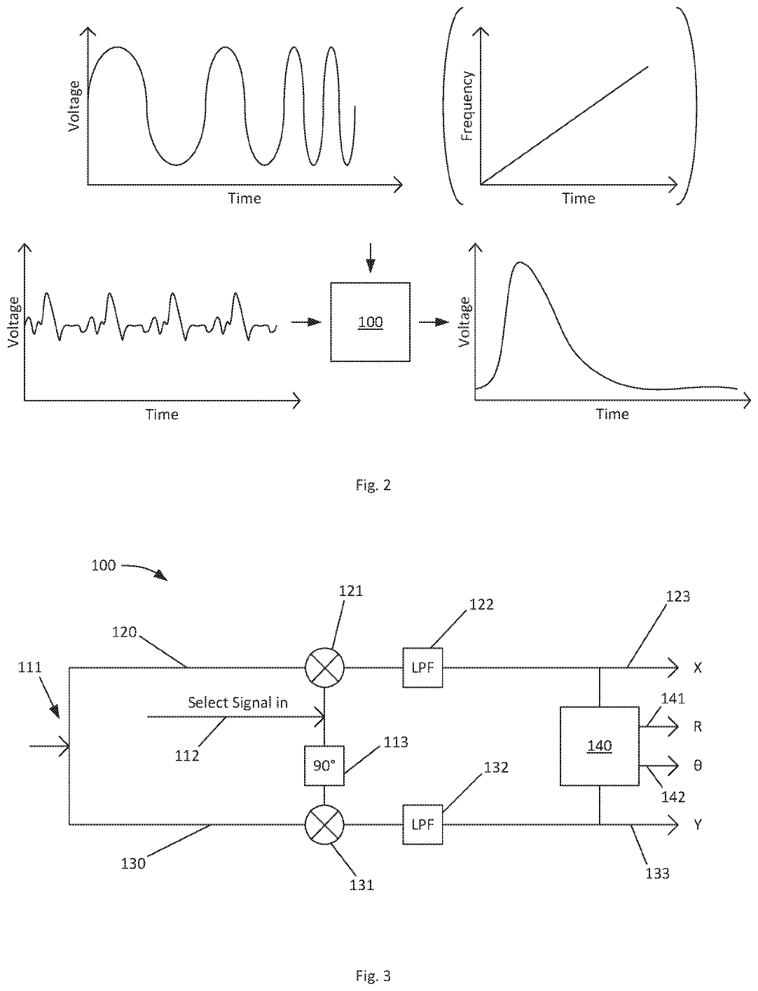

Example functionality provided by the LIA 100 will now be described with reference to FIG. 2.

FIG. 2 is a schematic diagram illustrating two inputs to the LIA 100, and a corresponding output from that LIA 100. The graph to the left of the LIA 100 illustrates a detection signal. The graph above the LIA 100 illustrates a selection signal. Both the detection signal and the selection signal are voltage signals. For ease of review, a frequency domain representation of the selection signal is shown in parentheses to the right of the voltage signal representation for the selection signal. The graph to the right of the LIA 100 shows an output from the LIA 100. This output is also shown as a voltage signal.

As shown, the selection signal is provided by a linear frequency sweep. The selection frequency increases linearly over time, so that the selection signal frequency provided to the LIA 100 increases (linearly) with time. A first time period is shown for the selection signal frequency. As shown, the linear frequency increase continues throughout this first time period. The detection signal is a mixed signal comprising a plurality of different components at different frequencies.

As the selection frequency provided to the LIA 100 changes, the output signal may also change. The output from the LIA 100 for a given selection frequency may provide an indication of a magnitude of the component of the detection signal at said selection frequency. For example, the LIA 100 may be a dual phase amplifier. The LIA 100 may also be configured to provide an indication of a phase of the component of the detection signal at the selection frequency.

The system is configured so that the magnitude of each beat frequency component within the detection signal may provide an indication of the number of sample light photons incident on the detector associated with that beat frequency. In particular, as each beat frequency corresponds to a time of flight difference between sample and reference light, the magnitude for each beat frequency may provide an indication of the number of detected sample light photons having that time of flight difference. In other words, a distribution of the magnitudes for each beat frequency component within the detection signal may provide an indication of a corresponding distribution of the times of flight associated with detected sample light photons. The specific relationship between frequency and time of flight depends on the characteristics of the sample light, specifically its wavelength-swept characteristics.

For example if the sample light frequency increases over time, beat frequency components at the lowest frequencies will correspond to the shortest sample light times of flight (as the difference between sample and reference light times of flight will be small). Conversely, beat frequency components at the highest frequencies will correspond to the longest sample light times of flight. For each of the beat frequency components, the magnitude associated with that particular beat frequency component (and its corresponding selection frequency) provides an indication of the number of sample light photons which had the time of flight associated with that beat frequency component. The distribution of the respective magnitude of each of the beat frequency components in the detection signal may provide an indication of a time of flight distribution (‘DTOF’) for the sample light photons. This is shown in FIG. 2.

The system is arranged so that, throughout the first time period, the selection frequency is swept so that it spans from a lowest frequency (initially) to a highest frequency (finally). The lowest selection frequency may be at or below a lowest beat frequency in the detection signal, and the highest selection frequency may be at or above the highest beat frequency in the detection signal. Each selection frequency between the highest and lowest frequencies corresponds to beat frequency (and thus time of flight) in the detection signal. The system is arranged so that, for the plurality of different beat frequency components within the detection signal which are at a beat frequency corresponding to a selection frequency provided to the LIA 100, an output from the LIA 100 for that selection frequency will provide an indication of the magnitude of that beat frequency component within the detection signal.

As will be clear to the person skilled in the art, the system 10 may easily be adapted to other frequency sweep patterns, for example monotonically decreasing frequency with time. In such an exemplary case, the lowest selection frequency may be at or above a highest beat frequency in the detection signal, and the highest selection frequency may be at or below a lowest beat frequency in the detection signal.

As can be seen in FIG. 2, the LIA 100 is configured to sweep the selection signal through a plurality of different selection frequencies (the frequency increases linearly in FIG. 2) so that the output signal contains an indication of the magnitude for each of a plurality of different times of flight for sample light photons (each associated with a corresponding beat frequency in the detection signal). The output signal shown in FIG. 2 is a voltage signal. In other words, the LIA 100 is configured to generate an output signal, which in this case an analogue output signal, that is indicative of the DTOF for the sample light.

Because the frequency spectrum of the output signal of the LIA 100 may be lower than the frequency spectra of the selection signal and the detection signal, embodiments may therefore reduce digitizer requirements for the imaging system. A digital representation of the sample light DTOF may be obtained without requiring as great a digitisation bandwidth. For example, the output from the LIA 100 may be provided to ADC 306 (e.g. as shown in Inset A of FIG. 1), optionally via an amplifier (e.g. amplifier 305). The digital representation of that LIA output signal may effectively provide DTOF data for the sample light. The system may be configured to provide imaging of the object based on said DTOF data. For instance, the system may be an iNIRS system configured to provide neuroimaging and analysis based on DTOF data for a subject's brain tissue. Operation of the LIA 100 of FIG. 2 will now be described in combination with operation of the iNIRS system of FIG. 1.

Wavelength-swept light from the light source 20 is directed towards the subject's scalp/object to be imaged (via sample delivery channel 25), and also to the detector 30 (via reference delivery channel 26 and reference receiving channel 36). Some of the light directed towards the subject's scalp from the light source 20 is received, as sample light, in the sample receiving channel 35 (where a portion of that sample light will have interacted with, e.g. scattered from, the subject's brain tissue). The detector 30 combines the sample light with the reference light (e.g. using combiner and splitter 301) to provide combined light signals containing components at a plurality of beat frequencies associated with differences in wavelength between the different components of sample light and the reference light. That combined light signal is then converted into an electrical signal to be provided to the LIA 100. In FIG. 1, the combined light signal may be split into two signals and provided to the balanced detector 303, which outputs a current signal to be converted into a voltage signal by TIA 304. The resulting voltage signal contains a plurality of beat frequency components which correspond to beat frequencies present in the combined light signal. The contribution of each of these beat frequency components to the voltage signal is influenced by the respective contribution of the beat frequency components in the combined light signal (and thus by the number of photons of sample light incident on the detector at each time of flight).

This voltage signal is provided to the LIA 100 as the detection signal. The system is also arranged to provide a selection signal to the LIA 100 (and to vary the selection frequency of that selection signal). For example, the system may include a local oscillator coupled to the LIA 100 and operable to provide selection signals to the LIA 100.

The selection signal provided to the LIA 100 may start with a low selection frequency (e.g. a frequency at or below the lowest beat frequency present in the voltage signal). Each time the selection signal is at a selection frequency which corresponds to (e.g. is the same as) a beat component frequency in the voltage signal, the LIA 100 will provide one or more output signals indicative of that beat component in the voltage signal. The LIA 100 is configured to combine the two input signals (e.g. as a mixer). The signal output may be low pass filtered (e.g. to remove higher frequency components from the output mixed signal). The resulting signal may be indicative of the beat frequency component that was in the detection signal. Based on this, an indication of the characteristics of the beat frequency component within the detection signal may be determined.

The LIA 100 will therefore provide an output signal containing information about a first beat frequency component in the voltage signal. The selection frequency is then changed (e.g. increased), and the process repeats, with the LIA 100 then providing an output signal containing information about second, third, fourth etc. beat frequency components within the voltage signal. Over time (e.g. during the first time period), the LIA 100 will have output a (possibly continuous) series of signals associated with each of a plurality of different selection frequencies, and these signals will be indicative of the plurality of different beat frequency components in the voltage signal. These output signals may provide information about the number of sample light photons received at the detector for each of a plurality of different times of flight (e.g. where each time of flight is associated with a corresponding beat frequency).

The ADC 306 may convert this voltage signal output from the LIA 100 into a digital signal, e.g. the ADC 306 may comprise an integrator (or low pass filter) for providing a digital representation of the voltage signal it receives. Such a digital output signal from the ADC 306 may therefore be indicative of DTOF data for the sample. That is, the distribution contained in the digital output signal may itself be representative of the sample light DTOF. The controller 40 may be configured to process such digital output signals from the ADC 306 to convert them into a DTOF distribution. For example, a known scaling (or mapping), e.g. as stored in a data store of the controller 40, may be applied to convert the digital signal representative of a voltage output from the LIA 100 into a corresponding distribution DTOF distribution. Neuroimaging and analysis of the subject's brain may be performed based on such DTOF distributions (e.g. in the manner described above).

A number of different example lock-in amplifier arrangements will now be described with reference to FIGS. 3 to 7.

Each of these lock-in amplifiers includes: a first input port arranged to receive a detection signal, and a second input port arranged to receive a selection signal. The first input port may be connected downstream of the light combiner and the conversion circuitry for converting the combined light signals into electrical signals (e.g. voltages) which are representative of those combined light signals. The second input port may be coupled to a local oscillator arranged to output electrical signals (e.g. voltages) at chosen selection frequencies. The local oscillator may be operable to vary the selection frequency of those selection signals.

Each LIA 100 may include one or more low pass filters. The LIA 100 may be configured to provide frequency mixing of the detection signal with the selection signal. The low pass filter may be arranged to provide low pass filtering of such a mixed signal. As will be appreciated in the context of the present disclosure, the mixed signal may contain higher and lower frequency combinations of the selection and detection signals. The low pass filter may be arranged to remove higher frequency components (e.g. to enable a lower frequency component to be extracted from the mixed signal).

The LIA 100 is configured to provide at least one output signal. Each output signal will be indicative of at least one property of the contribution to the detection signal at the selection frequency. The controller may be configured to determine an indication of the total number of photons incident on the detector associated with the beat frequency corresponding to that selection frequency based on this at least one output signal from the LIA 100.

The LIA 100 may have one or more output ports. Each output port may be configured to provide an output signal. Each output signal will be indicative of at least one property of the relevant component of the detection signal (i.e. the component at the selection frequency). The LIA 100 may be configured to provide an output signal indicative of at least one of: (i) a magnitude (‘R’) of the relevant component of the detection signal, (ii) a phase (‘θ’) of the relevant component of the detection signal, (iii) an in-phase component (‘X’) for the relevant component of the detection signal, and (iv) a quadrature component (‘Y’) for the relevant component of the detection signal. For example, the LIA 100 comprise at least one of: (i) a magnitude output port, (ii) a phase output port, (iii) an in-phase component output port, and (iv) a quadrature component output port.

At least one output signal from the LIA 100 may be indicative of a magnitude of the component of the detection signal at the selection frequency. The controller may be configured to determine an indication of the total number of photons incident on the detector associated with the beat frequency corresponding to that selection frequency based on this magnitude. For example, the larger the magnitude of the component in the detection signal at a given frequency, the greater the number of photons incident on the detector at a time of flight corresponding to that given frequency.

At least one output from the LIA 100 may be indicative of a phase of the component in the detection signal at the selection frequency. For example, the LIA 100 may be arranged to provide output signals in the form of a polar coordinate representation of the detection signal (e.g. containing magnitude and phase information). The polar representation (e.g. phase and magnitude) may be output from respective phase and magnitude output ports. Additionally, or alternatively, the LIA 100 may be configured to process in-phase and quadrature component output signals (e.g. from a respective in-phase component output port 123 and a quadrature component output port 124), and to determine phase and/or magnitude information therefrom. The LIA 100 may be configured to provide output in-phase and quadrature signals (e.g. and not provide phase and magnitude outputs).

The LIA 100 may be configured to provide the relevant output signal(s) for each different selection frequency applied to the LIA 100. In other words, for each different beat frequency which corresponds to a selection frequency applied to the LIA 100, the LIA 100 may output an indication of at least one property of the component of the detection signal at that beat frequency/selection frequency. That indication may be provided as an output from a relevant port of the LIA 100. For example, the output from the LIA 100 may be one or more pulses (e.g. a pulse pair) which repeats once per wavelength sweep for the selection frequency. For example, each output signal may be in the form of a voltage pulse. The shape of such a voltage pulse may be indicative of the one or more properties (e.g. magnitude) for each of the plurality of relevant components of the detection signal (i.e. those at frequencies corresponding to selection frequencies that were applied to the LIA 100). The controller may be configure to obtain DTOF data for the sample light based on a digital representation of such an output (e.g. each output pulse) from the LIA 100.

The LIA 100 may have one or more different output ports. In some examples, the LIA 100 has two output ports: either an in-phase component (‘X’) output port and a quadrature component (‘Y’) output port, or a magnitude (‘R’) output port and a phase (‘θ’) output port. In examples, where the X and Y output ports are used, the system may be configured to process these output signals to obtain R and θ information. In some examples, all four output ports may be provided. The LIA 100 may be provided in combination with ADC 306 and/or controller 40. In some examples, the output ports may each be configured to output analogue signals (e.g. voltage pulses). In other examples, output voltage pulses may be converted into digital signals, and one or more of the output signals may be digital. For example, R and e output signals may be obtained based on a digital representation of analogue output signals.

The LIA 100 may be a dual phase (dual channel) LIA. the LIA 100 may be configured to provide a polar coordinate output (e.g. to output R and θ signals for the relevant component of the detection signal), and/or to provide in-phase and quadrature component outputs (X and Y components). For example, the LIA 100 may include one or more phase shifting components. In particular, the LIA 100 may include one component configured to provide a 90° phase shift for one channel of the LIA 100. For example, the LIA 100 may be configured to shift the selection signal by 90° for one channel of the LIA 100 (but not for the other channel). One channel of the LIA 100 may then be mixing the detection signal with the selection signal, and the other channel of the LIA 100 may be mixing the detection signal with a 90° phase shifted version of the selection signal.

A first example LIA 100 will now be described with reference to FIG. 3.

FIG. 3 shows a lock-in amplifier (‘LIA’) 100. The LIA 100 has a first input port 111 and a second input port 112. The LIA 100 includes a first channel 120, a first mixer 121, and a first low pass filter 122. The LIA 100 also includes a phase shifter 113, as well as a second channel 130, a second mixer 131, and a second low pass filter 132. The LIA 100 has four output ports: an in-phase component output port 123, a quadrature component output port 124, a magnitude output port 141, and a phase output port 142. A processor 140 is also shown in FIG. 3, but it will be appreciated that this could be provided by e.g. ADC 306 and controller 40.

The LIA 100 forms part of signal processing circuitry associated with a detector 30. As shown in Inset A of FIG. 1, the detector 30 may have a light combiner for combining sample and reference light to provide combined light signals. Signal processing circuitry associated with that detector is arranged to convert the combined light signals into electrical signals and to process those electrical signals to obtain digital data indicative of the sample light times of flight. The LIA 100 may be arranged between circuitry for converting light signals into electrical signals and circuitry for converting electrical signals into digital data. For example, the LIA 100 may be located downstream of balanced detector 303 and TIA 304, and upstream of ADC 306.

The first input port 111 is coupled to the signal processing circuitry for receiving detection signals therefrom. The detection signals will typically be voltage signals. The second input port 112 may be coupled to a local oscillator (not shown) for receiving selection signals therefrom. The LIA 100 includes a first channel 120 and a second channel 130. The first channel 120 couples the first and second input ports 111, 112 to the in-phase component output port 123. The second channel 130 couples the first and second input ports 111, 112 to the quadrature component output port 124.

For the first channel 120, the first input port 111 and the second input port 112 are each coupled to the first mixer 121. The first mixer 121 is arranged to receive detection signals (from the first input port 111) and selection signals (from the second input port 112). The mixer is arranged to provide a mixer output in the form of a mixed signal. The mixer is arranged to provide heterodyne mixing between the detection signal from the first input port 111 and the selection signal from the second input port 112. The first mixer 121 is configured to provide a heterodyned combination of the detection signal and the selection signal so that the mixed signal output from the first mixer 121 contains a DC component corresponding to the combination of the selection signal at the selection frequency and the beat component of the detection signal at the selection frequency. The first mixer 121 may also output a component at double the selection frequency. The output 121 also comprises a component at a frequency corresponding to the phase offset between the detection signal and the selection signal (at that specific selection frequency). The first mixer 121 is coupled to the first low pass filter 122. The LIA 100 is arranged so that the output mixed signal from the first mixer 121 is low pass filtered by the first low pass filter 122. The first low pass filter 122 is arranged to remove higher frequency components from the output mixed signal. In particular, the low pass filter is arranged to remove the double selection frequency component (as well as other higher frequency components which may be present in the output mixed signal). The first low pass filter 122 is coupled to the in-phase component output port 123. The in-phase component output port 123 is arranged to receive the low pass filtered signal. This low pass filtered signal may be formed of a DC component having an amplitude which corresponds to the root mean square amplitude of the signal. In other words, the low pass filtered signal may provide an indication of the number of sample light photons that were incident on the detector which gave rise to a beat frequency at the selection frequency. This low pass filtered signal may be output from the in-phase component output port 123. That output signal may provide an indication of the in-phase component of the relevant component within the detection signal. The in-phase component (‘X’) may comprise the cosine of the phase difference between the selection signal and the detection signal and an amplitude related to the amplitude of the detection signal component at the selection frequency. For example, X=Re (Z)=R cos θ, where R is the root mean square amplitude of the signal and 0 is the phase of the relevant component of the detection signal relative to the selection signal.

The arrangement of the second channel 130 corresponds to that of the first channel 120, except the phase shifter 113 is also included to provide a 90° phase shift to the selection signal. In FIG. 3, the phase shifter 113 is arranged between the second input port 112 and the second mixer 131 (to provide a 90° phase shift to the selection signal). The second mixer 131 is arranged to receive the detection signal as one input and the 90° phase shifted selection signal as the other input, and to provide a mixed output signal based on the two input signals. That mixed output signal is filtered by the second low pass filter 132, and provided to the quadrature component output port 124. The quadrature component output port 124 provides an output indicative of the quadrature component (‘Y’) of the signal. For example, Y=Im (Z)=R sin θ, where R is the root mean square amplitude of the signal and θ is the phase of the relevant component of the detection signal relative to the selection signal.

In other words, the LIA 100 may be arranged to provide two output signals: one indicative of the in-phase component and another indicative of the quadrature component. In FIG. 3, the in-phase component output port is configured to provide an analogue signal representative of the in-phase component. The quadrature component output port 124 is configured to provide an analogue signal representative of the quadrature component. These two output ports may be coupled to ADC 306 for digitisation. For example, an amplifier may be provided to amplify each signal. Each signal may be provided to its own respective ADC channel, or they may be applied sequentially to the same ADC channel.

The LIA 100 of FIG. 3 is also configured to obtain an indication of the magnitude and phase of the signal (i.e. to extract R and θ). As will be appreciated in the context of the present disclosure, these may be obtained using relevant trigonometric relationships. For example, the processor 140 shown in FIG. 3 may be implemented digitally, e.g. so that the magnitude output port 141 and the phase output port are digital. For example, the X and Y component signals may be digitised and then processed digitally to obtain the corresponding indication of R and θ.

FIG. 4 shows another LIA 100. The LIA 100 has a first input port 111, a second input port 112, a mixer 151 and a low pass filter 152. The LIA 100 also includes ADC 306 and controller 40. As shown, the controller 40 comprises a phase shifter 41, processor 42, and a (digital) magnitude output port 4141 and a (digital) phase output port 4142.

The LIA 100 of FIG. 4 has a number of similarities to the LIA 100 of FIG. 3. Unlike the LIA 100 of FIG. 3, the LIA 100 of FIG. 4 has one mixer (mixer 151) and one low pass filter (low pass filter 152). For the LIA 100 of FIG. 4, the output ports of the LIA 100 are all provided in the digital domain.

As with the LIA 100 of FIG. 3, the LIA 100 of FIG. 4 is arranged to receive a detection signal at the first input port 111 and a selection signal at the second input port 112. The first and second input ports 111, 112 are coupled to the mixer 151. The mixer 151 is configured to receive the selection signal and the detection signal, and to provide a mixed signal as its output. The low pass filter 152 is coupled to the mixer 151. The low pass filter 152 is configured to low pass filter 152 the mixed signals output from the mixer 151. The low pass filter 152 is coupled to the ADC 306. The ADC 306 is configured to digitise the filtered mixed signals from the low pass filter 152.

The ADC 306 is configured to convert each input analogue signal (e.g. each voltage signal) into a digital representation of that signal. The controller 40 is coupled to the ADC 306 to receive digital signals therefrom. As shown in FIG. 4, the controller 40 is formed of phase shifter 41 and processor 42. The controller 40 is configured to receive a digital signal from the ADC 306 and to provide that digital signal to both the phase shifter 41 and the processor 42. The phase shifter 41 is configured to provide a 90° phase shift to the digital signal. For example, to provide the phase shifter 41, the controller 40 may be configured to perform a Hilbert transform on the digital signal. The processor 42 may be configured to process the digital signal and the 90° phase shifted digital signal to obtain magnitude (R) and phase (θ) components for the signal (e.g. as described above in relation to FIG. 3). Alternatively, X component at the output of the ADC and the Y component (after the 90° phase shift) may also be output by LIA 100.

In other words, the LIA 100 may be configured to use a mixer 151 and a filter to obtain initial mixed and filtered analogue signals. The system may be configured to convert each analogue signal into a digital representation thereof (e.g. using ADC 306), and to use digital signal processing (e.g. controller 40) to obtain magnitude and phase component outputs for each mixed and filtered analogue signal provided by the mixer 151.

FIG. 5 shows another LIA 100. The LIA 100 has a first input port 111 and a second input port 112. The LIA 100 includes a first channel 120, a first mixer 121, and a first low pass filter 122. The LIA 100 also includes a phase shifter 113, as well as a second channel 130, a second mixer 131, and a second low pass filter 132. The apparatus also includes a multiplexer 115, ADC 306, a controller 40 and digital magnitude output port 4141 and digital phase output port 4142.

The LIA 100 of FIG. 5 is similar to the LIA 100 of FIG. 3. ADC 306 and controller 40 are also shown in FIG. 5. The LIA 100 of FIG. 5 differs from that of FIG. 3 in that it also includes multiplexer 115. Each of the first low pass filter 122 and the second low pass filter 132 are coupled to the multiplexer 115. The multiplexer 115 is arranged to receive: first mixed and low pass filtered signals (e.g. analogue signals, such as voltages) from the first channel 120, and second mixed and low pass filtered signals from the second channel 130. The multiplexer 115 is coupled to the ADC 306. The multiplexer 115 is configured to provide first signals and second signals to the ADC 306 so that the two different signals are separately identifiable by the controller 40. The multiplexer 115 may be configured to alternate which signal it provides to the ADC 306. For example, the multiplexer 115 may be configured to provide time multiplexing so that a first signal is provided to the ADC 306 for a given time period, then a second signal is provided for another time period. The controller 40 may be configured to identify (and separate) the first and second signals digitally based on the time at which they were provided to the ADC 306. A single digitiser channel may be utilised to digitise signals from both the first and second channels. The controller 40 may be configured to process the digital signals obtained from the ADC 306 to obtain magnitude and phase information, e.g. as described above.

FIG. 6 shows another LIA 100. The LIA 100 has a first input port 111 and a second input port 112. The LIA 100 includes a first channel 120, a first mixer 121, and a first low pass filter 122. The LIA 100 also includes a phase shifter 113, as well as a second channel 130, a second mixer 131, and a second low pass filter 132. The LIA 100 includes a first junction 161, a second junction 162, a third mixer 163, a third low pass filter 164, and a phase output port 142. The LIA 100 also includes a first rectifier (shown as first diode 125), a second rectifier (show as second diode 135), a first integrator 126, a second integrator 136, an adder 171, a differentiator 172, and a magnitude output port 141.

FIG. 6 is similar to the LIA 100 of FIG. 3. The LIA 100 of FIG. 6 differs from that of FIG. 3 in that the LIA 100 of FIG. 6 is arranged to provide analogue output signals for phase and magnitude. As with the LIA 100 of FIG. 3, the LIA 100 has first and second channels, each of which is arranged to provide mixed and low pass filtered signals. The first low pass filter 122 is coupled to the first junction 161 and the second low pass filter 132 is coupled to the second junction 162. Each junction splits the signal processing circuitry between circuitry for obtaining the phase analogue signals and circuitry for obtaining the magnitude analogue signals.

For the phase analogue signals, the first and second junction 161, 162 each respectively couple the first low pass filter 122 and the second low pass filter 132 to the third mixer 163. The first and second junctions 161, 162 may be splitters. The third mixer 163 is arranged to receive two inputs: first mixed and low pass filtered signals (from the first mixer 121), and second mixed and low pass filtered signals (from the second mixer 131). The third mixer 163 is configured to provide an output signal to third low pass filter 164, and the third low pass filter 164 is configured to pass filter that output signal to provide an analogue phase signal. The low pass filter is coupled to the phase output port to provide analogue phase signals thereto.

For the magnitude analogue signals, the first and second junction 161, 162 each respectively couple the first low pass filter 122 and the second low pass filter 132 to the first diode 125 and the second diode 135. The first diode 125 is coupled to the first integrator 126, and the first integrator 126 is coupled to the adder 171. The second diode 135 is coupled to the second integrator 136, and the second integrator 136 is coupled to the adder 171. Each diode is arranged to rectify the mixed and low pass filtered signal it receives to provide rectified signals to its connected integrator. Each integrator is arranged to integrate the rectified signals it receives and to output the integrated signals to the adder 171. The adder 171 is arranged to receive two inputs: the integrated signals from each integrator, and to provide an added signal output to the differentiator 172. The differentiator 172 is configured to differentiate the added signal to provide the analogue magnitude output signal. The differentiator 172 is coupled to the magnitude output port 141 to provide analogue magnitude output signals thereto.

An ADC may be coupled to each of the phase output port and the magnitude output port 141. The ADC is configured to generate digital phase and magnitude signals. For example, each output port may be coupled to its own digitiser channel, and/or the LIA 100 may include a multiplexer arranged for the phase and magnitude analogue signals to be digitised using a single digitiser channel.

Each LIA 100 of the present disclosure is configured to receive two inputs: a selection signal at a selection frequency, and a detection signal comprising a plurality of different beat frequency components. The LIA 100 is configured to combine the selection and detection signals to use the selection signal to select the beat frequency component from the detection signal which is at the selection frequency. Each of the selection and the detection signals may be analogue signals (e.g. voltages). The LIA 100 may be arranged to output one or more analogue signals or to output digital signals. For example, the LIA 100 may operate in combination with ADC 306 and controller 40 to generate digital output signals, or the LIA 100 may generate analogue output signals which may then be digitised and processed by the ADC 306 and controller 40. In either case, the controller 40 may be configured to provide digital signal processing and/or analysis of digital signals representative of the LIA 100 output (e.g. information about the in-phase component, the quadrature component, the magnitude and/or phase of the received signals). In other words, the controller 40 may be configured to process output signals from the LIA 100, e.g. for providing neuroimaging and analysis.

Additionally, or alternatively, to controlling processing of output signals from the LIA 100, the controller 40 may be configured to control operation of the LIA 100. In particular, the controller 40 may be configured to control the application of the selection signal to the LIA 100. For example, the LIA 100 may be coupled to a local oscillator, where that oscillator is operable to provide the selection signals to the LIA 100 at different selection frequencies. The controller 40 may be configured to select which selection frequency is applied, and/or to control how the selection frequency to be applied is varied. The controller 40 may be configured to control the selection signal to sweep through a plurality of selection frequencies during the first time period. For each selection frequency applied, the LIA 100 may be configured to output a corresponding beat frequency component. The controller 40 may be configured to vary the selection frequency so that it increases or decreases through the first time period.

For each selection signal to be applied to the LIA 100, the controller 40 may be configured to select the selection frequency of that selection signal and/or to select for how long that selection signal is to be applied to the LIA 100. For each selection frequency applied to the LIA 100, the LIA 100 may provide an output signal indicative of a beat frequency component of the detection signal at that selection frequency. As will be appreciated in the context of the present disclosure, the shorter the amount of time for which each selection signal is applied, the greater the number of different beat frequency components which may identified in a given time period. As will also be appreciated in the context of the present disclosure, the longer the amount of time for which each selection signal is applied, the better the frequency resolution for each individual beat frequency component identified in the detection signal. As such, there may be a trade-off between sampling rate and frequency resolution for the system.

The controller 40 may be configured to vary the selection frequencies and the amount of time for which each selection signal is applied to the LIA 100 to provide chosen characteristics for sampling rate/frequency resolution of the system. For example, the controller 40 may be configured to control operation of the LIA 100 to be in either: (i) a higher resolution mode in which selection signals may be applied to the LIA 100 for a longer duration (and thus in which the sampling rate is lower), and (ii) a higher sampling rate mode in which selection signals may be applied to the LIA 100 for a shorter duration (and thus in which the resolution is lower). The controller 40 may be configured to switch between the two modes of operation, e.g. in response to obtaining an indication that higher resolution or high sampling rate is required.

The controller 40 may be configured to control operation of the LIA 100 to provide a linear selection frequency sweep (e.g. in which the selection frequency is linearly increased or decreased over time) for at least a portion of the first time period. For example, the linear sweep may occur throughout the entirety of the first time period.

The controller 40 may be configured to vary the selection frequency sweep to be non-linear for at least a portion of the first time period. For example, the controller 40 may control operation so that the speed with which selection frequencies are swept varies. For a quicker sweep, a greater range of selection frequencies will be covered per unit time. As such, a broader range of selection frequencies may be sampled, but the frequency resolution for these may be lower. In other words, a quicker sweep may increase the measurement speed (e.g. as it will take less time to cover all of the intended selection frequencies), but in so doing, this may sacrifice the frequency resolution to some extent (e.g. it may be a quicker/lower resolution mode of operation). Conversely, for a slower sweep, a smaller range of selection frequencies is used per unit time. The slower sweep may provide a higher resolution mode of operation. The controller 40 may have an indication of selection frequencies of interest, e.g. selection frequencies having associated beat frequencies which may be of particular use for imaging the object. The controller 40 may be configured to control operation so that the speed with which the selection frequency is swept is lower when the selection frequencies are within a range of the selection frequencies of interest. For example, the controller 40 may be configured to control the selection frequency sweep rate to be lower when in a selection frequency range of interest, thereby to provide a higher resolution for the beat frequencies of interest, but a lower resolution (and thus quicker measurement) for other frequency ranges (e.g. which are less likely to contain any beat frequencies of interest). Frequency resolution may thus be improved for the most relevant beat frequency components without needing to provide as large a drop in sampling rate to achieve this increased frequency resolution. Another arrangement for varying the operational characteristics of an imaging system will now be described with reference to FIG. 7.

FIG. 7 shows a lock-in amplifier arrangement 100. The LIA arrangement 100 includes four LIAs (first LIA 101, second LIA 102, third LIA 103 and fourth LIA 104). Each of the four LIAs has a first input port 111. The first LIA has a second input port 112a. The second LIA has a second input port 112b. The third LIA has a third input port 112. The fourth LIA has a fourth input port 112d. ADC 306/Controller 40 is also shown in FIG. 7.

Each of the LIAs may be of any suitable type disclosed herein. Each LIA will output an indication of the beat frequency component corresponding to the selection frequency applied to that LIA. In the arrangement 100 of FIG. 7, there are a plurality of (e.g. four) LIAs which are coupled to the detector to receive the same detection signal. In other words, each of the LIAs is arranged to receive the same detection signal. The LIAs differ in that they are each arranged to receive different selection signals. For example, each LIA may be coupled to its own oscillator, which outputs an associated selection signal at a chosen selection frequency. The LIAs are arranged to receive selection signals in different selection frequency ranges. Each LIA may receive selection signals with selection frequencies which span through a different frequency range. The different selection frequency ranges may be chosen to span the expected beat frequency range. In other words, across the four different LIAs, the frequency range for selection frequencies may cover an expected frequency range for the beat frequencies. Each individual LIA may have an associated selection frequency range which spans a portion of this beat frequency spectrum, but where the combined selection frequency range spanned by all of the LIAs covers the majority of, or the entire, expected beat frequency spectrum.

For instance, Inset B in FIG. 7 shows an expected distribution to be obtained by the LIAs. The frequency range for the distribution is split into four (equal) portions: A, B, C and D. The arrangement 100 is configured for each of the four LIAs to receive selection frequencies in a different one of these portions. The output from each individual LIA may therefore be indicative of a different portion of the beat frequency spectrum. The controller 40 may be configured to process the output signals from the different LIAs and to provide imaging across the beat frequency spectrum. The four different outputs may be combined to provide a single beat frequency distribution (which spans a larger frequency range than that of the output from any individual LIA). The arrangement 100 of FIG. 7 may enable the sampling rate to be increased without needing to compromise on frequency resolution (or vice-versa).

Different example LIAs have been described herein. It is to be appreciated that features of these LIAs may be combined with features of the other LIAs. For example, LIAs may be dual channel or single channel. LIAs may at least partially be implemented in the digital domain, or they may be entirely implemented in the analogue domain. Each LIA may utilise one or more multiplexers for selectively outputting signals to a single ADC channel. LIAs may have any combination of the different output ports. For example, each LIA may have one or more output ports, e.g. each LIA could have one, two, three or four output ports. The output ports may be for any or all of in-phase components, quadrature components, phase and/or magnitude.

It will be appreciated in the context of the present disclosure that examples described herein are not intended to be limiting. Instead, examples describe certain potential ways of implementing the claimed technology. For example, the iNIRS system 10 is described with a series of optical cables providing channels and probes for coupling those channels to the subject's scalp. However, it will be appreciated that the probes themselves may be part of the optical channels, or probes may not be provided at all. Similarly, the arrangement of reference channels is just intended to show that reference light is delivered from the light source to the light detectors via optical channels (rather than via the subject's brain tissue). For example, each light source may include one reference channel for each light detector, where that reference channel directly connects the light source to the light detector. In which case, there may be no reference connections in the system at all. Alternatively, and as shown in FIG. 1, the reference light may be transmitted on a common reference optical channel, where some of that reference light is taken from the common reference optical channel to each of the detectors. The light source may also be arranged to deliver light to one of a plurality of different locations on the subject's scalp. For example, the light source may be coupled to a plurality of different sample delivery channels, each extended towards the subject's scalp (e.g. from a light splitter). Multiple light sources may be provided. For example, each detector may be arranged to receive: (i) second sample light which was emitted by the second light source and directed towards the object to be imaged, and (ii) second reference light from the second light source via a second reference channel.

It will be appreciated that the particular arrangement shown for signal processing circuitry of the detector need not be considered limiting. Each light detecting arrangement 130 is configured to combine sample and reference light to provide a combined light signal with components at one or more beat frequencies, and to process those combined light signals to determine one or more properties of the subject's brain tissue. Any suitable signal processing and/or conversion circuitry could be used for this. For example, a transimpedance amplifier may not be needed (e.g. depending on the photodetector/ADC, no current to voltage conversion may be needed, or this may be performed in a different way). Similarly, a balanced photodetector need not be used, and instead a single photodetector, such as a photodiode, could be used. Similarly, the arrangement with the ADC shown in the Figs. need not be considered limiting. For example, multiple ADCs may be used (e.g. one for each detector output stream), or all detector output streams may be fed into one common ADC.

It will be appreciated from the discussion above that the examples shown in the figures are merely exemplary, and include features which may be generalised, removed or replaced as described herein and as set out in the claims. With reference to the drawings in general, it will be appreciated that schematic functional block diagrams are used to indicate functionality of systems and apparatus described herein. In addition the processing functionality may also be provided by devices which are supported by an electronic device. It will be appreciated however that the functionality need not be divided in this way, and should not be taken to imply any particular structure of hardware other than that described and claimed below. The function of one or more of the elements shown in the drawings may be further subdivided, and/or distributed throughout the apparatus of the disclosure. In some examples the function of one or more elements shown in the drawings may be integrated into a single functional unit.

As will be appreciated by the skilled reader in the context of the present disclosure, each of the examples described herein may be implemented in a variety of different ways. Any feature of any aspects of the disclosure may be combined with any of the other aspects of the disclosure. For example method aspects may be combined with apparatus aspects, and features described with reference to the operation of particular elements of apparatus may be provided in methods which do not use those particular types of apparatus. In addition, each of the features of each of the examples is intended to be separable from the features which it is described in combination with, unless it is expressly stated that some other feature is essential to its operation. Each of these separable features may of course be combined with any of the other features of the examples in which it is described, or with any of the other features or combination of features of any of the other examples described herein. Furthermore, equivalents and modifications not described above may also be employed without departing from the invention.

Certain features of the methods described herein may be implemented in hardware, and one or more functions of the apparatus may be implemented in method steps. It will also be appreciated in the context of the present disclosure that the methods described herein need not be performed in the order in which they are described, nor necessarily in the order in which they are depicted in the drawings. Accordingly, aspects of the disclosure which are described with reference to products or apparatus are also intended to be implemented as methods and vice versa. The methods described herein may be implemented in computer programs, or in hardware or in any combination thereof. Computer programs include software, middleware, firmware, and any combination thereof. Such programs may be provided as signals or network messages and may be recorded on computer readable media such as tangible computer readable media which may store the computer programs in non-transitory form. Hardware includes computers, handheld devices, programmable processors, general purpose processors, application specific integrated circuits (ASICs), field programmable gate arrays (FPGAs), and arrays of logic gates.

Any controller of the present disclosure may be implemented with fixed logic such as assemblies of logic gates or programmable logic such as software and/or computer program instructions executed by a processor. The controller may comprise a central processing unit (CPU) and associated memory, connected to a graphics processing unit (GPU) and its associated memory. Other kinds of programmable logic include programmable processors, programmable digital logic (e.g., a field programmable gate array (FPGA), a tensor processing unit (TPU), an erasable programmable read only memory (EPROM), an electrically erasable programmable read only memory (EEPROM), an application specific integrated circuit (ASIC), or any other kind of digital logic, software, code, electronic instructions, flash memory, optical disks, CD-ROMs, DVD ROMs, magnetic or optical cards, other types of machine-readable mediums suitable for storing electronic instructions, or any suitable combination thereof. In particular, any controller of the present disclosure may be provided by an ASIC.

Other examples and variations of the disclosure will be apparent to the skilled addressee in the context of the present disclosure.

1. An imaging system comprising:
a light source configured to provide wavelength-swept emission of light;
a sample delivery channel coupled to the light source and arranged to be coupled to an object to be imaged to direct light from the light source towards said object;
a reference channel coupled to the light source for receiving light therefrom;
a sample receiving channel arranged to be coupled to the object to be imaged for receiving sample light from the object;
a detector coupled to the sample receiving channel for receiving sample light, and coupled to the reference channel for receiving reference light, wherein the detector is arranged to combine the received sample light with the reference light to provide a combined light signal comprising one or more components at a beat frequency between sample light and reference light; and
a lock-in amplifier arranged to: (i) receive a detection signal based on the combined light signal and formed of one or more components at different frequencies, (ii) receive a selection signal at a selection frequency, and (iii) to provide one or more output signals indicative of a component of the detection signal at the selection frequency;
wherein the imaging system is arranged to control the selection signal to be at each of a plurality of different selection frequencies during a first time period; and
wherein the imaging system is configured to provide imaging of the object based on the output signals from the lock-in amplifier.

2. The system of claim 1, wherein the lock-in amplifier comprises a dual phase lock-in amplifier.

3. The system of claim 2, wherein the lock-in amplifier comprises:
a first input port arranged to receive the detection signal;
a second input port arranged to receive the selection signal;
a first output port arranged to provide an output indicative of an in-phase component of the detection signal; and
a second output port arranged to provide an output indicative of a quadrature component of the detection signal.

4. The system of claim 2 or 3, wherein the lock-in amplifier further comprises at least one of:
a third output port arranged to provide an output indicative of a magnitude of the detection signal; and
a fourth output port arranged to provide an output indicative of a phase associated with the detection signal.

5. The system of claim 1 claim, further comprising a controller configured to control the selection signal applied to the lock-in amplifier.

6. The system of claim 5, wherein the controller is configured to control the selection signal to sweep through a plurality of selection frequencies during the first time period.

7. The system of claim 6, wherein the controller is configured to control at least a portion of the frequency sweep of the selection signals to be non-linear.

8. The system of claim 7, wherein the controller is configured to control the selection frequency sweep to sweep through a first frequency range at a greater speed than for a second frequency range.

9. The system of claim 5, wherein the controller is configured to control the selection signal applied to the lock-in amplifier so that, during at least a portion of the first time period, the selection signal is a linearly-chirped sinusoidal signal.

10. The system of claim 5, wherein the controller is configured to control operation of the system to switch between a higher resolution mode and a lower resolution mode.

11. The system of claim 10, wherein the controller is configured to apply a slower frequency sweep for the selection signal when operating in the higher resolution mode than when operating in the quicker mode.

12. The system of claim 11, wherein the controller is configured to provide a higher sweep rate for sweeping through the selection frequencies when operating in the lower resolution mode.

13. The system of claim 1, wherein the system comprises two or more lock-in amplifiers.

14. The system of claim 13, wherein each of the lock-in amplifiers is arranged to operate in a different portion of a frequency spectrum.

15. The system of claim 14, wherein each of the lock-in amplifiers is arranged to sweep its selection signals through a different portion of the frequency spectrum, and wherein the system is configured to image the object based on results from each of the lock-in amplifiers.

16. The system of claim 1, wherein the detection signal and the selection signal are analogue signals.

17. The system of claim 16, wherein the detection signal and the selection signal are voltage signals.

18. The system of claim 16 further comprising one or more photo detectors arranged to provide the detection signal to the lock-in amplifier, wherein said photo detectors are arranged to convert the combined light signal into an electrical signal to provide the detection signal.

19-23. (canceled)

24. An imaging method comprising:
providing wavelength-swept emission of light from a light source;
directing light from the light source: (i) towards an object to be imaged, and (ii) to a reference channel;
receiving, at a detector, reference light from the reference channel and sample light from the object to be imaged, wherein the received sample light comprises light delivered towards the object from the light source;
optically combining sample light with reference light to provide a combined light signal comprising one or more components at a beat frequency between sample light and reference light;
providing, to a lock-in amplifier: (a) a detection signal based on the combined light signal and formed of one or more components at different frequencies, and (b) a selection signal at a selection frequency, and wherein the lock-in amplifier is configured to provide one or more output signals indicative of the component of the detection signal at the selection frequency;
controlling the selection signal to be at each of a plurality of different selection frequencies during a first time period;
providing imaging of the object based on the output signals from the lock-in amplifier.

25. A computer program product comprising computer program instructions configured to control operation of an imaging system to perform the method of claim 24.
